(19) United States
(12) Patent Application Publication
(10) Pub. No.: US 2011/0112132 A1
(43) Pub. Date: May 12, 2011

(54) MEANS FOR THE TREATMENT OF ACUTE AND CHRONIC DISORDERS OF CEREBRAL CIRCULATION, INCLUDING INSULT, BASED ON HYDROGENATED PYRIDO (4,3-B) INDOLES (VARIANTS), PHARMACOLOGICAL MEANS BASED THEREON AND METHOD FOR THE USE THEREOF

(75) Inventors: Sergey Olegovich Bachurin, Chernogolovka (RU); Taisiya Leonovna Garibova, Moscow (RU); Tatiana Alexandrovna Voronina, Moscow (RU); Vladimir Viktorovich Grigoriev, Chernogolovka (RU)

(73) Assignee: Medivation Neurology, Inc.

(21) Appl. No.: 12/518,090

(22) PCT Filed: Nov. 30, 2007
(86) PCT No.: PCT/US07/24626

§ 371 Date: Jan. 7, 2011

(30) Foreign Application Priority Data
Dec. 7, 2006 (RU) 2006143332

(51) Int. Cl. A61K 31/437; A61P 9/10

(52) U.S. Cl. 514/292

(57) ABSTRACT

A means for the treatment of insult based on hydrogenated pyrido(4,3-b)indoles (variants) of formula (1) or formula (2) a pharmacological means based thereon and a method for the use thereof relate to the use of chemical compounds in the field of medicine and may be used for the treatment of ischemic and hemorrhagic insults and their consequences.

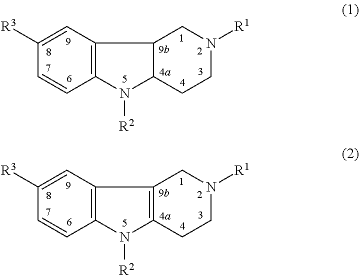

### CROSS-REFERENCE TO RELATED APPLICATIONS

This application claims priority to Russian Patent Application No. 2006143332, filed Dec. 7, 2006, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The invention relates to the field of medicine, and specifically to the use of chemical compounds, such as hydrogenated pyrido[4,3-b]indoles or pharmaceutically acceptable salts thereof, with the object of creating therapeutic means for the treatment of ischemic and hemorrhagic insults and their consequences.

### BACKGROUND OF THE INVENTION

Acute insufficiency or disturbance of cerebral circulation and ischemic and hemorrhagic insults are among the most widespread vascular pathologies, which often lead to disability and noticeably increase the mortality rate. Insult may cause injury to and the death of significant areas of the brain, as a consequence of which impairment of cognitive functions, depression and disorientation develop in addition to neurological deficit (paresis, paralysis) in patients who have suffered an insult (E. I. Gusev and V. I. Skvortsova, in Cerebral ischemia, Moscow, Meditsina, 2001, p. 238; R. G. Robinson, “The clinical neuropsychiatry of stroke,” in Cognitive, behavioral and emotional disorders following vascular brain injury (1998) (Cambridge University Press, 1998, p. 563)).

Modern pharmacology has a fairly extensive arsenal of means which act on various stages of the cascade of pathological processes during insult. The treatment of insult is directed at restoration of arterial patency (tissue activator), and prevention of thrombogenesis (fibrinolytics, anticoagulants, antiaggregants) and the death of viable neurons. Cerebrolysin, choline alfoscerate, carnitine chloride, mexidol and glycine are prescribed to prevent neuron death in the “ischemic shadow” region.

The “ischemic shadow” region refers to the peripheral field surrounding the infarction focus. Blood flow to brain tissue in that region is reduced but not stopped, allowing the neurons to survive but not to perform their normal functions. Successful therapies for treatment of ischemic insults treat the infarct while also restoring function of neural tissue in the “ischemic shadow” region, thereby reducing the size of the resulting infarct. Unsuccessful therapies do not restore function of neural tissue in the “ischemic shadow” region, resulting in the massive death of neurons and glial cells, thereby increasing size of the resulting infarct.

Certain vasoactive preparations (vinopocetine, nicergoline, cinnarizin) also have a protective effect in treating ischemic insult, and are prescribed with the object of increasing the blood supply to the ischemized tissue. However, in this case one cannot exclude the “robbing” phenomenon, which is manifested as a reduction in blood flow in the ischemic zone due to enhancement of blood flow in healthy tissues (E. I. Gusev and V. I. Skvortsova, in Cerebral Ischemia, Moscow, Meditsina, 2001, p. 238). Furthermore, treatment with these preparations is insufficiently effective, and the consequences of hemorrhagic insult are particularly resistant to treatment. Standard treatments typically attempt to support function of vital organs and to restore homeostasis.

The search which is currently in progress for effective drugs for the treatment of insult among calcium and sodium channel antagonists, antagonists of NMDA receptors, positive modulators of AMPA receptors, and substances having antiradical activity has not yet led to tangible results (mainly due to the pronounced side-effects and the high toxicity of the compounds) (H. Brauner-Osborne, J. Egebjerg, E. Nielsen, U. Madsen and P. Krogsgaard-Larsen, “Ligands for glutamate receptors: design and therapeutic prospects,” J. Med. Chem. 48(14):2609-2645 (2000); J. Hatton, “Pharmacological Treatment of Traumatic Brain Injury: Review of Agents in Development,” CNS Drugs 15(7):553-581 (2001)).

Among the calcium channel antagonists, the L-type calcium channel blocker nimodipine is often employed, particularly to treat both ischemic and hemorrhagic insults. At the same time, preparations of this group have significant side-effects and disadvantages, one of which is the presence of cardiovascular effects, leading to “robbing” of the brain (Register of Drugs in Russia, Encyclopedia of Drugs, 14th ed. (Ed. G L Vyshkovskiy, Moscow, RLS, 2006)).

Certain known compounds, including derivatives of tetra- and hexa-hydro-1H-pyrido[4,3-b]-indole, exhibit a wide spectrum of biological activity. For example, in the series of 2,3,4,5-tetrahydro-1H-pyrido[4,3-b]indoles, the following types of activity have been observed: antihistamine (OS-DE No. 1813229 of 6 Dec. 1968, No. 1952800 of 20 Oct. 1969), central-depressive and anti-inflammatory activity (U.S. Patent

No. 3,718,657, issued 13 December 1970), neuroleptic activity (C. A. Herbert, S. S. Plattner, and W. N. Welch (1980) Mol. Pharm., 17(1):38-42) and others. Derivatives of 2,3,4,4a,5,9b-hexahydro-1H-pyrido[4,3-b]indole exhibit psychotropic (W. N. Welch, C. A. Herbert, A. Weissman, and K. B. Koe (1986) J. Med. Chem. 29(10):2093-2099), antidepressive, antiarrhythmic and other types of activity.

As described in U.S. Pat. No. 6,187,785 (“the '785 patent”) and U.S. Pat. No. 7,021,206 (“the '206 patent”), hydrogenated pyrido[4,3-b]indole derivatives such as dimebon have NMDA antagonist properties, which make them useful for treating neurodegenerative diseases, such as Alzheimer's disease. Dimebon can be useful for treating Alzheimer's disease and other neurodegenerative diseases both alone (as described in the '785 patent and the '206 patent) and in combination with other compounds (as described in a PCT application claiming priority to U.S. Provisional Patent Application No. 60/854,866, filed Oct. 26, 2007). As described in WO 2005/055951, hydrogenated pyrido[4,3-b]indole derivatives, such as dimebon, are useful as human or veterinary geroprotectors, e.g., by delaying the onset and/or development of an age-associated or related manifestation and/or pathology or condition, including disturbance in skin-hair integument, vision disturbance and weight loss. As described in U.S. patent application Ser. No. 11/543,529 (U.S. Patent Publication No. 2007-0117835-A1) and Ser. No. 11/543,341 (U.S. Patent Publication No. 2007-0117834-A1), hydrogenated pyrido[4,3-b]indole derivatives such as dimebon are useful as neuroprotectors for use in treating and/or preventing and/or slowing the progression or onset and/or development of Huntington's disease. As described in WO 2007/087425, published Aug. 2, 2007, hydrogenated pyrido[4,3-b]indole derivatives such as dimebon are useful for treating schizophrenia. As described in WO 2007/020516, filed Sep. 20, 2007, hydrogenated pyrido[4,3-b]indole derivatives such as dimebon are useful for treating amyotrophic lateral sclerosis.

There remains a significant medical need for additional or alternative therapies for treating acute insufficiency of cerebral circulation and ischemic and hemorrhagic insults. Preferably, the therapeutic agents can limit the extent of disability, improve the quality of life, reduce impairment of cognitive function, and/or prolong the survival time for patients suffering from such injuries.

The task, to the solution of which the invention now proposed is directed, is to extend the arsenal of means which can be utilized as new effective drugs for the treatment of insult—where cerebral can be one of the most serious and least amenable to treatment vascular affections of the brain.

### DETAILED DESCRIPTION OF THE INVENTION

### Definitions

As used herein, unless clearly indicated otherwise, the terms “a,” “an,” and the like refer to one or more. It is also understood and clearly conveyed by this disclosure that reference to “the compound” or “a compound” includes and refers to any compound or pharmaceutically acceptable salt or other form thereof as described herein, such as the compound dimebon.

As used herein, the term “insult” refers to two broad classes of insult: “ischemic insult” and “hemorrhagic insult.” The terms “ischemic insult” and “hemorrhagic insult” refer to any of a number of pathological conditions resulting from disturbance of blood flow, including cerebral ischemia or infarction and ischemic stroke (resulting from an abrupt decrease in blood flow to the brain) and cerebral, subcranial and ventricular hemorrhage. The term also refers to mixed-type insults with combined ischemic and hemorrhagic foci. Cerebral ischemia or ischemic stroke results from blockage of a blood vessel in the brain, which cuts off blood flow to part of the brain. Strokes are caused by, among other things, formation of a blood clot inside an artery of the brain (i.e., a thrombotic stroke), formation of a blood clot elsewhere in the body that travels to an artery in the brain (i.e., an embolic stroke), acute transient cerebral blood circulation disturbances, or rupture of a blood vessel in the brain (i.e., a hemorrhagic stroke). Clinical manifestations of ischemic stroke are displayed as focal symptoms prevailing over general cerebral symptoms, and include partial paralysis, numbness, apraxia (inability to perform learned movements), and loss of vision, as well as various cognitive defects including perceptual disorders and speech problems.

As used herein, unless clearly indicated otherwise, the term “an individual” refers to a mammal, including but not limited to a human. The individual may be a human who has been diagnosed with or is suspected of having suffered an ischemic or hemorrhagic insult. The individual may be a human who exhibits one or more symptoms associated with ischemia or hemorrhagic insult. The individual may be a human who has a mutated or abnormal gene associated with elevated risk of ischemic or hemorrhagic insult but who has not been diagnosed with such an injury. The individual may be a human who is genetically or otherwise predisposed to developing an ischemic or hemorrhagic insult.

In one variation, the individual is a human who has not been diagnosed with and/or is not considered at risk for developing Alzheimer's disease, Huntington's disease, amyotrophic lateral sclerosis, or schizophrenia. In one variation, the individual is a human who does not have impaired cognition associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof.

As used herein, an “at risk” individual is an individual who is at risk of developing or suffering an ischemic or hemorrhagic insult. An individual “at risk” may or may not have detectable disease, and may or may not have displayed detectable disease prior to the treatment methods described herein. “At risk” denotes that an individual has one or more so-called risk factors, which are measurable parameters that correlate with likelihood of experiencing an ischemic or hemorrhagic insult. An individual having one or more of these risk factors has a higher probability of suffering such an injury than an individual without those risk factor(s). Risk factors include, but are not limited to, age, sex, race, diet, history of previous disease or injury, presence of precursor disease or injury, genetic (i.e., hereditary) considerations, and environmental exposure. Individuals at risk for ischemic or hemorrhagic insult include, e.g., those having relatives who have experienced such injuries, and those whose risk is determined by analysis of genetic or biochemical markers.

As used herein, the term “pharmaceutically active compound,” “pharmacologically active compound” or “active ingredient” refers to a chemical compound, such as a hydrogenated pyrido(4,3-b)indole, that induces a desired effect, e.g., treating and/or preventing and/or delaying the onset or severity of ischemic or hemorrhagic insult.

As used herein, the term “pharmacological means” or “pharmaceutical formulation” refers to the use of any therapeutic dosage form, including immediate or sustained release forms, containing a compound, e.g., a compound of formula (1) or formula (2), which may find prophylactic or therapeutic use in medicine for the treatment of ischemic or hemorrhagic insult. Such means or formulations may also contain pharmaceutically acceptable excipients, including preservatives, solubilizers, stabilizers, re-wetting agents, emulgators, sweeteners, dyes, adjusters, salts for the adjustment of osmotic pressure, buffers, coating agents or antioxidants.

As used herein, the term “pharmaceutically acceptable” or “pharmacologically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “effective amount” refers to the use of that amount of compound, e.g., a compound of formula (1) or formula (2) which in combination with its activity and toxicity characteristics, and also on the basis of the knowledge of a specialist, should be effective in a given therapeutic form.

As used herein, the term “therapeutically effective amount” refers to an amount of a compound or a combination therapy sufficient to produce a desired therapeutic outcome (e.g., reducing the severity or duration of, stabilizing the severity of, or eliminating one or more symptoms associated with ischemic or hemorrhagic insult). For therapeutic use, beneficial or desired results include, e.g., clinical results such as reducing or eliminating inflammation associated with ischemic or hemorrhagic insult, improving cognition or otherwise reversing cognitive impairment, decreasing one or more symptoms resulting from the disease or injury (biochemical, histologic and/or behavioral), including associated complications and intermediate pathological phenotypes presenting during development or progression of ischemic or hemorrhagic insult, increasing the quality of life of those suffering such injuries, decreasing the dose of other medications required to treat the insults, enhancing effect of another medication, and/or prolonging survival of patients.

A “prophylactically effective amount” refers to an amount of a compound or a combination therapy sufficient to prevent or reduce the severity of one or more future symptoms of ischemic or hemorrhagic insult when administered to an individual who is susceptible and/or who may develop such insults. For prophylactic use, beneficial or desired results include, e.g., results such as eliminating or reducing the risk, lessening the severity, or delaying the onset of the insult, including biochemical, histologic and/or behavioral symptoms of ischemic or hemorrhagic insult, its complications and intermediate pathological phenotypes presenting during development and/or progression of the disease.

As used herein, “treatment” or “treating” is an approach for obtaining beneficial or desired results, including clinical results. For purposes of this invention, beneficial or desired clinical results include, but are not limited to, one or more of the following: decreasing one more symptoms resulting from ischemic or hemorrhagic insult, limiting the extent of disability resulting from ischemic or hemorrhagic insult, increasing the quality of life, reducing any impairment of cognitive function, decreasing the dose of one or more other medications required to treat the disease or injury, and/or prolonging survival time for individuals suffering from such injuries. In some embodiments, an individual or combination therapy of the invention reduces the severity of one or more symptoms associated with ischemic or hemorrhagic insult by at least 10, 20, 30, 40, 50, 60, 70, 80, 90, or 95% compared to the corresponding symptom in the same subject prior to treatment or compared to the corresponding symptom in other subjects not receiving the therapy.

As used herein, the term “combination therapy” refers to a first therapy that includes one or more hydrogenated pyrido[4,3-b]indoles or pharmaceutically acceptable salts thereof in conjunction with a second therapy that includes one or more other compounds (or pharmaceutically acceptable salts thereof) or therapies (e.g., surgical procedures) useful for decreasing one more symptoms resulting from ischemic or hemorrhagic insult, limiting the extent of disability resulting from ischemic or hemorrhagic insult, increasing the quality of life, reducing any impairment of cognitive function, decreasing the dose of one or more other medications required to treat the disease or injury, and/or prolonging survival time for individuals suffering from such injuries. Administration in “conjunction with” another compound includes administration in the same or different composition, either sequentially, simultaneously, or continuously using the same or different route of administration for each compound. In some variations, the combination therapy optionally includes one or more pharmaceutically acceptable carriers or excipients, non-pharmaceutically active compounds, and/or inert substances.

As used herein, the term “simultaneous administration” means that a first therapy and a second therapy of a combination therapy are administered with a time separation of no more than about 15 minutes, such as no more than about any of 10, 5, or 1 minutes. When the compounds are administered simultaneously, the first and second therapies may be contained in the same composition (e.g., a composition comprising both a hydrogenated pyrido[4,3-b]indole and the L-type calcium channel blocker nimodipine) or in separate compositions (e.g., a hydrogenated pyrido[4,3-b]indole is contained in one composition and nimodipine is contained in another composition).

As used herein, the term “sequential administration” means that the first therapy and second therapy in a combination therapy are administered with a time separation of more than about 15 minutes, such as more than about any of 20, 30, 40, 50, 60 or more minutes. Either therapy may be administered first. The first and second therapies are contained in separate compositions, which may be contained in the same or different packages or kits.

Thus, an effective amount of a combination therapy includes an amount of the first therapy and an amount of the second therapy that when administered sequentially, simultaneously, or continuously produces a desired outcome. Suitable doses of any of the co-administered compounds may optionally be lowered due to the combined action (e.g., additive or synergistic effects) of the compounds. In various embodiments, treatment with the combination of the first and second therapies may result in an additive or even synergistic (e.g., greater than additive) result compared to administration of either therapy alone. In some embodiments, a lower amount of each pharmaceutically active compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a small amount of pharmaceutically active compound results in a reduction in the number, severity, frequency, or duration of one or more side-effects associated with the compound.

As is understood in the clinical context, an effective dosage of a drug, compound or pharmaceutical composition containing a compound described by the invention, e.g., a compound of the formula (1) or (2) or any compound described herein (e.g., any of compounds 1 to 9) may be achieved in conjunction with another drug, compound or pharmaceutical composition that contains one or more compounds that restore arterial patency, prevent thrombogenesis (e.g., fibrinolytics, anticoagulants, antiaggregants), minimize or prevent the death of viable neurons (e.g., cerebrolysin, choline alfoscerate, carnitine chloride, mexidol and glycine), increase blood flow to the ischemized tissue (e.g., vasoactivators such as vinopocetine, nicergoline, cinnarizin), antagonize calcium and/or sodium channels (e.g., the L-type calcium channel blocker nimodipine), antagonze NMDA receptors, and modulate AMPA receptors.

As used herein, the term “controlled release,” “sustained release,” or “delayed release” refers to a drug-containing formulation or fraction thereof in which release of the drug is not immediate, i.e., with a “controlled,” “sustained,” or “delayed release” formulation, administration does not result in immediate release of the drug into an absorption pool. In certain embodiments, the compound is administered to the individual as a sustained release form or as part of a sustained release system, such as a system capable of sustaining the rate of delivery of a compound to an individual for a desired duration, which may be an extended duration such as a duration that is longer than the time required for a corresponding immediate-release dosage form to release the same amount (e.g., by weight or by moles) of compound, and can be hours or days. A desired duration may be at least the drug elimination half-life of the administered compound and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

### Exemplary Hydrogenated pyrido(4,3-b)indoles

Hydrogenated pyrido([4,3-b])indoles of formula (1) or formula (2) can be used to treat ischemic or hemorrhagic insult.

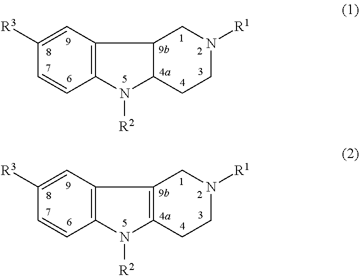

When compounds of formula (1) are used, R¹ is selected from the group containing CH₃—, CH₃CH₂— or PhCh₂; R² is selected from the group containing H, PhCH₂ or 6-CH₃-3-Py-(CH₂)₂—; and R³ is selected from the group containing H, CH₃— or Br—. Those compounds may comprise salts with pharmaceutically acceptable acids.

One of the compounds which may be used as a means for the treatment of insult may be a compound of formula (1) in which R¹ corresponds to CH₃—, R² is H—, and R³ is CH₃—. This compound may be in the form of the (±)cis-isomer. When compounds of formula (2) are used. R¹ is selected from the group containing CH₃—, CH₃CH₂— or PhCh₂—; R² is selected from the group containing H—, PhCH₂— or 6-CH₃-3-Py-(CH₂)₂—, and R³ is selected from the group containing H—, CH₃— or Br—. Said compounds may comprise salts with pharmaceutically acceptable acids.

One of the compounds which may be used as a means for the treatment of insult may be a compound of formula (2) in which R¹ corresponds to CH₃CH₂— or PhCH₂—, R² corresponds to H—, and R³ is H—; or a compound where R¹ corresponds to CH₃—, R² corresponds to PhCH₂—, and R³ is CH₃—; or a compound where R¹ corresponds to CH₃—, R² corresponds to 6-CH₃-3-Py-(CH₂)₂—, and R³ is H—; or a compound where R¹ corresponds to CH₃—, R² corresponds to 6-CH₃-3-Py-(CH₂)₂—, and R³ is CH₃—; or a compound where R¹ corresponds to CH₃—, R² corresponds to H—, and R³ is H— or CH₃—; or a compound where R¹ corresponds to CH₃—, R² corresponds to H—, and R³ is Br—. In one variation, the compound is dimebon. Any of the compounds indicated above may be used as a means for the treatment of insult.

Known compounds of formula (1) and (2) are widely used in pharmacological practice. Extensive investigations have been carried out into a series of known compounds which comprise derivatives of tetra- and hexa-hydro-1H-pyrido([4,3-b]-indole and which exhibit a wide spectrum of biological activity. The following types of activity were found in the 2,3,4,5-tetrahydro-1H-pyrido[4,3-b]indole series: antihistamine (OS-DE No. 1813229 of 6 Dec. 1968, No. 1952800 of 20 Oct. 1969), central-depressant, anti-inflammatory (U.S. Pat. No. 3,718,657, issued 13 Dec. 1970), neuroleptic (C. A. Herbert, S. S. Plattner, and W. N. Welch (1980) Mol. Pharm., 17(1):38-42) and others. Derivatives of 2,3,4,4a,5,9b-hexahydro-1H-pyrido[4,3-b]indole exhibit psychotropic (W. N. Welch, C. A. Herbert, A. Weissman, and K. B. Koe (1986) J. Med. Chem. 29(10):2093-2099), antidepressive, antiarrhythmic and other types of activity.

All the above-mentioned compounds are known from the literature and include the following specific compounds:
1. cis(±)2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]-indole;
4. 2,8-dimethyl-5-benzyl-2,3,4,5-tetrahydro-1H-pyrido[4,3-b]indole and its hydrochloride;
5. 2-methyl-5-[2-(6-methyl-3-pyridyl)ethyl]-2,3,4,5-tetrahydro-1H-pyrido[4,3-b]indole and its sesquisulfate monohydrate;
6. 2,8-dimethyl-5[2-(6-methyl-3-pyridyl)ethyl]-2,3,4,5-tetrahydro-1H-pyrido[4,3-b]indole and its dihydrochloride (dimebon).
7. 2-methyl-2,3,4,5-tetrahydro-1H-pyrido[4,3-b]-indole;
8. 2,8-dimethyl-2,3,4,5-tetrahydro-1H-pyrido[4,3-b]-indole and its methyl iodide;
9. 2-methyl-8-bromo-2,3,4,5-tetrahydro-1H-pyrido-[4,3-b]indole and its hydrochloride;

The preparation and neuroleptic properties of compound 1 are known, for example, from the publication L. N. Yakhontov and R. G. Glushkov (1983) “Synthetic Medicinal Drugs,” (Ed. by A. G. Natradze, Moscow, Meditsina) pp. 234-237. The preparation of compounds 2, 8 and 9, and also information that they possess the properties of serotonin antagonists, is described, for example, by C. J. Cattanach, A. Cohen and B. H. Brown (1968) J. Chem. Soc. Series C:1235-1243. The synthesis of compound 3 is described, in particular, in the paper by N. P. Buu-Hoi, O. Roussel and P. Jacquignon (1964) J. Chem. Soc. No. 2, pp. 708-711. In 1956, N. F. Kucherova and N. K. Kochetkov described the synthesis of compound 4 in Obshchey khimii 26:3149-3154, while the preparation of compounds 5 and 6 is known, for example, from the 1973 paper by A. N. Kost, M. A. Yurovskaya and T. V. Mel'nikova in Khimiya geterotsiklicheskikh soedineniy, No. 2, pp. 207-212. The synthesis of compound 7 was described in 1954 by U. Horlein in Chem. Ber., Bd 87, hft 4, pp. 463-472, while in 1981 M. A. Yurovskaya and I. L. Rodionov described the preparation of the methyl iodide of compound 8 in Khimiya geterotsiklicheskikh soedineniy, No. 8, pp. 1072-1078.

A number of therapeutic preparations based on derivatives of tetra- and hexa-hydro-1H-pyrido[4,3-b]-indole are manufactured: diazoline (mebhydroline), dimebon, dorastine, carbidine (dicarbine), stobadine, hevotroline. Diazoline (2-methyl-5-benzyl-2,3,4,5-tetrahydro-1H-pyrido[4,3-b]indole dihydrochloride (M. A. Klyuev, “Drugs used in USSR medical practice,” Moscow, Meditsina 1991, p. 512), dimebon (2,8-dimethyl-5-(2-(6-methyl-pyridyl-3)ethyl)-2,3,4,5-tetrahydro-1H-pyrido[4,3-b]indole) (M. D. Mashkovskiy, “Medicinal drugs,” part 1 of 2, 12th ed., Moscow, Meditsina, 1993, p. 383), and also its close analog dorastine (2-methyl-8-chloro-5-(2-(6-methyl-3-pyridyl)-ethyl)-2,3,4,5-tetrahydro-1H-pyrido[4,3-b]indole dihydrochloride (USAN and USP Dictionary of Drug Names (United States Adopted Names 1961-1988, current U.S. Pharmacopeia and National Formulary for Drugs, and Other Non-proprietary Drug Names,” 1989, 26th Edition, p. 196) are known as antihistamine preparations. Carbidine (dicarbine) (cis(±)2,8-dimethyl-2,3,4,4a,5,9b-hexahydro-1H-pyrido[4, 3-b]indole dihydrochloride) is a Russian-produced neuroleptic with an antidepressive effect (L. N. Yakhontov and R. G. Glushkov, “Synthetic medicinal drugs,” (Ed. A. G. Natradze, Moscow, Meditsina, 1983, pp. 234-237), while its (−)-isomer, stobadine, is known as an anti-arrhythmic drug (M. Kitlova, P. Gibela, and J. Drimal (1985) Bratisl. Lek. Listy 84(5):542-546); hevotroline (8-fluoro-2-(3-3-pyridyl)-propyl)-2,3,4,5-tetrahydro-1H-pyrido[4,3-b]indole) is an antipsychotic and anxiolytic drug (M. Abou-Gharbi, U. R. Patel, M. B. Webb, J. A. Moyer and T. H. Ardnee (1987) J. Med. Chem. 30:1818-1823).

It has recently been found that derivatives of hydrogenated pyrido[4,3-b]indoles of formula (1) or (2), particularly dimebon, are capable of acting on the two main subtypes of inotropic glutamate receptors of the mammalian CNS—AMPA and NMDA receptors, which allows them to be employed as means for the treatment of Alzheimer's Disease and as geroprotectors. Dimebon potentiates the transmembrane currents induced by the activation of AMPA receptors, and simultaneously blocks the NMDA receptors (V. V. Grigor'eve, O. A. Dranyy and S. O. Bachurin, “A comparative study of the mechanism of action of the preparations dimebon and memantine on the AMPA and NMDA subtypes of glutamate receptors of rat brain neurons,” (2003) Bull. Exper. Biol. Med. No. 11, pp. 535-538).

The inventors have unexpectedly found that compounds of the invention, e.g., compounds of formula (1) and formula (2) have the ability significantly to eliminate the consequences of both hemorrhagic and ischemic insults. That ability differs from previously known properties of hydrogenated pyrido(4,3-b)indoles, and was not expected from prior characterizations of such compounds (in particular, as positive modulators of AMPA receptors or blockers of NMDA receptors) and may be employed as a therapeutic means for the treatment of insult.

According to the invention, a pharmacological means for the treatment of ischemic or hemorrhagic insult, containing an active principle and a pharmaceutically acceptable carrier, contains as the active principle an effective amount of a hydrogenated pyrido(4,3-b)indole e.g., a compound of formula (1) or formula (2).

In order to prepare a pharmacological means, one or several compounds of formula (1) or formula (2) are mixed as the active ingredient with a pharmaceutically acceptable carrier, known in medicine, in accordance with methods adopted in pharmaceuticals. The carrier may have various forms, depending on the therapeutic form of the preparation.

In accordance with the invention, a method for the treatment of ischemic or hemorrhagic insult comprises administering to a patient a pharmacological means containing an effective amount of a hydrogenated pyrido(4,3-b)indole of formula (1) or formula (2), such as dimebon, in a dose of 0.01-10 mg/kg of body weight at least once daily for a period necessary to achieve a therapeutic effect. The invention further provides methods for the treatment of ischemic or hemorrhagic insult comprising administering to a patient a pharmaceutical means containing an effective amount of a hydrogenated pyrido(4,3-b)indole of formula (1) or formula (2), wherein the hydrogenated pyrido(4,3-b)indole is compound 1, compound 2, compound 3, compound 4, compound 5, compound 6, compound 7, compound 8, or compound 9, or a pharmaceutically acceptable salt thereof, in a dose of 0.01-10 mg/kg of body weight at least once daily for a period necessary to achieve a therapeutic effect. In certain embodiments, the pharmaceutical means is administered intravenously at doses ranging from 0.15 to 0.3 mg/kg one or more times daily for a period necessary to achieve a therapeutic effect. In certain embodiments, the pharmaceutical means is administered orally in doses of 5-20 mg from one to three times daily for a period necessary to achieve a therapeutic effect.

In certain embodiments, the pharmaceutical means containing an effective amount of a hydrogenated pyrido(4,3-b)indole of formula (1) or formula (2), such as dimebon, is administered in combination with a second therapy that includes one or more other compounds (or pharmaceutically acceptable salts thereof) or therapies (e.g., surgical procedures) useful for decreasing one or more symptoms resulting from ischemic or hemorrhagic insult, limiting the extent of disability resulting from ischemic or hemorrhagic insult, increasing the quality of life, reducing any impairment of cognitive function, decreasing the dose of one or more other medications required to treat the disease, and/or prolonging survival time for individuals suffering from such injuries. In certain embodiments, the pharmaceutical means containing an effective amount of a hydrogenated pyrido(4,3-b)indole of formula (1) or formula (2), wherein the hydrogenated pyrido(4,3-b)indole is compound 1, compound 2, compound 3, compound 4, compound 5, compound 6, compound 7, compound 8, or compound 9, or a pharmaceutically acceptable salt thereof, is administered in combination with a second therapy that includes one or more other compounds (or pharmaceutically acceptable salts thereof) or therapies (e.g., surgical procedures) useful for decreasing one or more symptoms resulting from ischemic or hemorrhagic insult, limiting the extent of disability resulting from ischemic or hemorrhagic insult, increasing the quality of life, reducing any impairment of cognitive function, decreasing the dose of one or more other medications required to treat the disease, and/or prolonging survival time for individuals suffering from such injuries.

### Exemplary Formulations

One or more compounds of formula (1) or formula (2) can be used in the preparation of a formulation, such as a pharmaceutical formulation, by combining the compound or compounds as active ingredient with a pharmaceutically acceptable carrier, which are known in the art. See, e.g., Remington's Pharmaceutical Sciences, 20th ed. (2000), Mack Publishing Co., Philadelphia, Pa., which is incorporated herein by reference. Depending on the therapeutic form of the system (e.g., intravenous injection versus oral tablet), the carrier may be in various forms.

Pharmaceutical formulations may be administered in the form of conventional oral compositions, such as tablets, coated tablets, gelatin capsules with hard and soft coating, emulsions or suspensions. Preferably, however, they have liquid forms, suitable for intravenous injections or for droppers. Examples of carriers which can be utilized for the manufacture of such compositions are lactose, maize starch or its derivatives, talc, stearic acid or its salts, etc. Acceptable carriers for gelatin capsules with a soft coating are, for example, vegetable oils, waxes, fats, semi-solid and liquid polyols, etc. In addition, pharmaceutical preparations may contain preservatives, solubilizers, stabilizers, wetting agents, emulsifiers, sweeteners, colorants, correctives, salts for altering osmotic pressure, buffers, coating agents or antioxidants. They may also contain other substances which have desirable therapeutic properties. Preparative forms may comprise the normal standard dose and may be prepared by methods well known in pharmacy. Suitable formulations can be found, e.g., in Remington's Pharmaceutical Sciences, supra, which is incorporated herein by reference.

### Exemplary Dosing Regimens

For use herein, unless clearly indicated otherwise, a compound or combination therapy of the invention may be administered to the individual by any available dosage form. In one variation, the compound or combination therapy is administered to the individual as a conventional immediate release dosage form. In one variation, the compound or combination therapy is administered to the individual as a sustained release form or part of a sustained release system, such as a system capable of sustaining the rate of delivery of a compound to an individual for a desired duration, which may be an extended duration, such as a duration that is longer than the time required for a corresponding immediate-release dosage form to release the same amount (e.g., by weight or by moles) of compound or combination therapy, and can be hours or days. A desired duration may be at least the drug elimination half life of the administered compound or combination therapy and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The compound or combination therapy may be formulated for any available delivery route, whether immediate or sustained release, including an oral, mucosal (e.g., nasal, sublingual, vaginal, buccal or rectal), parenteral (e.g., intramuscular, subcutaneous, or intravenous), topical or transdermal delivery form. A compound or combination therapy may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), .pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The amount of compound, such as dimebon or any of compounds 1 to 9, in a delivery form may be any effective amount, which may be from about 10 ng to about 1,500 mg or more of the single active ingredient compound of a monotherapy or of more than one active ingredient compound of a combination therapy. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg.

The compound, such as dimebon or any of compounds 1 to 9, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosing frequency can be about a once weekly dosing. The dosing frequency can be about a once daily dosing. The dosing frequency can be more than about once weekly dosing. The dosing frequency can be less than three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

In one variation, dimebon is administered in a dose of 5 mg once a day. In one variation, dimebon is administered in a dose of 5 mg twice a day. In one variation, dimebon is administered in a dose of 5 mg three times a day. In one variation, dimebon is administered in a dose of 10 mg once a day. In one variation, dimebon is administered in a dose of 10 mg twice a day. In one variation, dimebon is administered in a dose of 10 mg three times a day. In one variation, dimebon is administered in a dose of 20 mg once a day. In one variation, dimebon is administered in a dose of 20 mg twice a day. In one variation, dimebon is administered in a dose of 20 mg three times a day. In one variation, dimebon is administered in a dose of 40 mg once a day. In one variation, dimebon is administered in a dose of 40 mg twice a day. In one variation, dimebon is administered in a dose of 40 mg three times a day.

### Exemplary Kits

The invention further provides kits comprising one or more compounds as described herein. The kits may employ any of the compounds disclosed herein and instructions for use. In one variation, the kit employs dimebon. In other variations, the kit comprises one or more of compounds 1 to 9. The compound may be formulated in any acceptable form. The kits may be used for any one or more of the uses described herein, and, accordingly, may contain instructions for any one or more of the stated uses (e.g., decreasing one more symptoms resulting from ischemic or hemorrhagic insult, limiting the extent of disability resulting from ischemic or hemorrhagic insult, increasing the quality of life, reducing any impairment of cognitive function, and/or prolonging survival time for individuals suffering from ischemic or hemorrhagic insult).

Kits generally comprise suitable packaging. The kits may comprise one or more containers comprising any compound described herein, in unit dosage form or in multiple dosage form. Each component (if there is more than one component) can be packaged in separate containers or some components can be combined in one container where cross-reactivity and shelf life permit. The kit components can be supplied as liquids or powders. If supplied as powders, the kits may further comprise a pharmaceutically acceptable buffer or other solution for preparing a liquid formulation of the compound.

The kits may optionally include instructions, generally written instructions, although electronic storage media (e.g., magnetic diskette or optical disk) containing instructions are also acceptable, relating to the use of component(s) of the kit in methods of the present invention (e.g., methods of treating ischemic or hemorrhagic insult).

The instructions included with the kit generally include, for example, information describing the components of the kit and methods of administering those components to an individual in need thereof.

The technical result which can be secured when implementing the invention is a significant reduction in the mortality of patients, and reduction of the serious consequences of insult (paralyses, pareses, impairment of cognitive functions).

The possibility of implementing the invention with achievement of the stated object and securement of the technical result is confirmed, but not exhausted, by the following examples.

### EXAMPLES

### Example 1

This example describes a study of the anti-ischemic action of dimebon using a rat brain model of ischemia, produced by irreversible occlusion of the carotid arteries.

The therapeutic preparation Dimebon, 2,8-dimethyl-5-[2-(6-methyl-pyridyl-3-)ethyl]2,3,4,5-tetrahydro-1H-pyrido[4,3-b]indole dihydrochloride of formula (2):

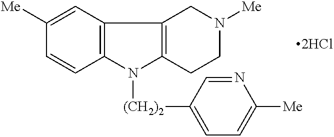

was selected as a representative of compounds of general formula (1) and (2).

Rat brain ischemia, produced by irreversible occlusion of the carotid arteries, was performed in accordance with “Methodological instructions for the experimental study of preparations for the treatment of cerebral circulation and migraine,” in “Handbook on the experimental (preclinical) study of new pharmacological substances,” Meditsina, Moscow, 2005, pp. 332-338.

Experiments were performed on cross-bred male white rats weighing 200-250 g, anesthetized with chloral hydrate (350 mg/kg, i/p). Irreversible single-step bilateral ligation of the common carotid arteries was performed on the animals. In the group of sham-operated animals, the ligatures were applied to the vessels but were not tightened.

After completing the operation, the animals were divided randomly into groups: group one rats were given dimebon intraperitoneally at 0.1 mg/kg administered 30 minutes after the ligature was tied, then daily for 14 days after operation; group two rats were given nimodipine intraperitoneally at 0.1 mg/kg administered 30 minutes after the ligature was tied, then daily for 14 days after operation. Group one and group two animals were experiencing an acute cerebral circulation disturbance at the time of drug administration. Control group and sham-operated animals were given equivalent volumes of physiological saline (0.9% sodium chloride) at the same times.

The data were processed statistically with the aid of the Biostat program, using parametric and nonparametric methods.

Recording the death of rats showed that no deaths had occurred after 24 hours in the group of sham-operated animals, while ischemia caused the death of 23.1% of rats in the first 24 hours, and 30.8% by day 14 after operation.

In the group of rats treated with dimebon, this figure was 7.7% over the entire period of observation, i.e., a statistically reliable reduction was observed in the number of rats which had died (Table 1). This testifies to the protective effect of dimebon in relation to the stringent index of the death of rats after irreversible occlusion of the carotid arteries.

In a similar dose of 0.1 mg/kg, nimodipine had a lesser ability to reduce death of the animals. 14 days after operation, the percentage death of rats did not differ essentially from the figure for the control animals (Table 1).

Dimebon thus exerted an anti-ischemic, anti-insult effect in experiments on animals with ischemic insult induced by irreversible occlusion of the carotid arteries, facilitating survival of the rats, which testifies to its anti-insult effect.

TABLE 1
Effect of intraperitoneal administration of dimebon on
the survival rate of animals with cerebral ischemia induced
by occlusion of the carotid arteries in rats

| | | 24 hours after | | 14 days after | |
|---|---|---|---|---|---|
| | | operation | | operation | |
| | | Number of animals dying during 14 days after | | | |
| | | ischemia relative to total number of | | | |
| Groups of | Doses, | operated animals in absolute units and in % | | | |
| animals | mg/kg | a.u. | % | a.u. | % |
| Sham- | | 0/14 | 0 | 0/14 | 0 |
| operated | | | | | |
| Ischemia | | 3/13 | 23.1* | 4/13 | 30.8* |
| Ischemia + | 0.1 | 1/13 | 7.7^{#} | 1/13 | 7.7^{#} |
| dimebon | | | | | |
| Ischemia + | 0.1 | 1/10 | 10^{#} | 2/10 | 20 |
| Nimodipine | | | | | |

Reliability of the differences between the group of sham-operated animals and rats with ischemic insult:
*is P ≦ 0.05(χ²); and between rats with ischemic insult and animals which received the preparations,
^{#}is P ≦ 0.05(χ²).

Reliability of the differences between the group of sham-operated animals and rats with ischemic insult: * is P≦0.05(χ²); and between rats with ischemic insult and animals which received the preparations, # is P≦0.05(χ²).

Neurological deficit in animals with cerebral ischemia induced by ligation of the carotid arteries was determined using the McGraw Stroke index as modified by I. V. Gannushkina (Functional angioarchitectonics of the brain (1977) (Moscow, Meditsina) p. 224). The severity of the condition was determined from the sum of the corresponding scores. The number of rats with mild symptoms up to 2.5 points on the Stroke-index scale (sluggish movements, limb weakness, hemiptosis, tremor, circular movements) and with severe manifestations of neurological impairment (from 3 to 10 points)—limb paresis, paralysis of lower limbs, lateral position, was noted.

Almost all the rats in the group of animals with ischemic insult exhibited neurological deviations, characterized by sluggish, weak and slow movements, hemiptosis and ptosis, which were particularly pronounced in the first days. On the third day of observation, those manifestations were slightly decreased; on the seventh day, they were reduced to a greater degree; and on the fourteenth day of observation, they completely disappeared (Table 2). Dimebon administered intraperitoneally at a dose of 0.1 mg/kg prevented the development of neurological deficit in rats with ischemia, statistically reliably reducing the number of animals with slowness of movements and bilateral hemiptosis when recording the indices on the first, and particularly on the seventh day after operation. Nimodipine administered intraperitoneally at a dose of 0.1 mg/kg produced no actual effect on neurological deficit indices in rats on the first day of observation, reduced the number of animals with unilateral hemiptosis on the third day of observation, and significantly diminished the neurological deficit in rats on the seventh day of observation (Table 2). By fourteen days after operation, no pathological signs were observed in either group. Pathological signs that were evaluated included: (1) sluggish, slow or weak movements; (2) limb weakness; (3) unilateral hemiptosis; (4) bilateral hemiptosis; and (5) unilateral ptosis.

Dimebon thus exerts a positive protective effect, reducing the symptoms of neurological deficit in rats one and seven days after cerebral ischemia induced by ligation of the carotid arteries, and in relation to this effect is superior to the action of nimodipine.

TABLE 2
Effect of intraperitoneal administration of dimebon
(0.1 mg/kg) on neurological deficit in rats after
ischemic insult, using the McGraw scale

| | Number of animals with neurological deficit, % | | | | |
|---|---|---|---|---|---|
| | sluggish, | limb | | | |
| Group of | slow | weak- | unilateral | bilateral | unilateral |
| animals | movements | ness | hemiptosis | hemiptosis | ptosis |
| 1 day | | | | | |
| Sham- | 10 | 0 | 0 | 0 | — |
| operated | | | | | |
| insult | 60.0 | 20.0 | 20.0 | 50.0 | — |
| insult + | 33.3* | 16.7 | 16.7 | 33.3* | — |
| dimebon | | | | | |
| insult + | 50 | 20 | 20 | 50 | — |
| nimodipine | | | | | |
| 3 days | | | | | |
| Sham- | 0 | 0 | 0 | 0 | 0 |
| operated | | | | | |
| insult | 33.3 | 22.2 | 11.1 | 44.4 | 11.1 |
| insult + | 33.3 | 16.7 | 0 | 25.0* | — |
| dimebon | | | | | |
| insult + | 30 | 0 | 20 | 20* | — |
| nimodipine | | | | | |
| 7 days | | | | | |
| Sham- | 0 | 0 | 0 | 0 | — |
| operated | | | | | |
| insult | 22.2 | 11.1 | 11.1 | 44.4 | — |
| insult + | 9.1* | 9.1 | 0 | 9.1* | — |
| dimebon | | | | | |
| insult + | 0 | 10 | 0 | 20* | — |
| nimodipine | | | | | |
| 14 days | | | | | |
| Sham- | 0 | 0 | 0 | 0 | 0 |
| operated | | | | | |
| insult | 0 | 0 | 0 | 0 | 0 |
| insult + | 0 | 0 | 0 | 0 | 0 |
| dimebon | | | | | |
| insult + | 0 | 0 | 0 | 0 | 0 |
| nimodipine | | | | | |

*Reliability of differences between rats with ischemia (control) and animals with ischemia which had received the preparations (P ≦ 0.05) (χ²).

### Example 2

A study of the anti-insult action of dimebon using an intracerebral post-traumatic hematoma (hemorrhagic insult) model.

The study was performed in accordance with the “Methodological instructions for the experimental study of preparations for the treatment of cerebral circulation and migraine,” in “Handbook on the experimental (preclinical) study of new pharmacological substances,” Meditsina, Moscow, 2005, pp. 332-338, as modified by A. N. Makarenko et al. (“Method for modeling local hemorrhage in various brain structures in experimental animals,” Zh. vyssh. nervn. deyat. (2002) 52(6):765-768).

The experiments were performed on cross-bred male white rats weighing 200-250 g, kept in a vivarium with free access to food (standard pelleted feed) and water, and with natural alternation of day and night. Using a special device (mandrin-knife) and stereotaxis, brain tissue of rats anesthetized with nembutal (40 mg/kg, i/m) was destroyed in the region of the capsule interna, with subsequent (after 2-3 minutes) introduction into the damage site of blood taken from under the rat's tongue (0.02-0.03 ml). Scalping and trepanning of the skull were performed on sham-operated animals.

The animals were divided into 4 groups: sham-operated, a group of animals with hemorrhagic insult, animals with hemorrhagic insult which received dimebon intraperitoneally at a dose of 0.1 mg/kg, and animals with hemorrhagic insult which received nimodipine intraperitoneally at a dose of 0.1 mg/kg. The effects of the substances were recorded 24 hours, and 3, 7 and 14 days after operation.

Dimebon and nimodipine were administered intraperitoneally to animals with insult in an identical dose of 0.1 mg/kg 3-3.5 hours after operation, and then daily for 14 days after operation. An equal volume of physiological saline was administered intraperitoneally to the control groups of animals at identical intervals. Each group consisted of 9-18 animals at the start of the experiment.

The neurological deficit in the animals was determined using the McGraw Stroke index as modified by I. V. Gannushkina (Functional angioarchitectonics of the brain (1977) (Moscow, Meditsina) p. 224). The severity of the condition was determined from the sum of the corresponding scores. The number of rats with mild symptoms up to 2.5 points on the Stroke-index scale (sluggish movements, limb weakness, unilateral hemiptosis, tremor, circular movements) and with severe manifestations of neurological impairment (from 3 to 10 points)—limb paresis, paralysis of lower limbs, lateral position, was noted.

Rat deaths were recorded over the entire 14 day period of observation.

The data were processed statistically with the aid of the Biostat program, using parametric and nonparametric methods. Nimodipine (in a dose of 0.1 mg/kg) was employed as the standard, using the scheme described above.

Recording the death of rats showed that the death of only 6.2% of the animals was observed by day 14 in the group of sham-operated animals, while this figure was 55.6% in the group with hemorrhagic insult, more than 33.3% of the animals dying in the first three days (Table 3).

Intraperitoneal dimebon at a dose of 0.1 mg/kg almost completely prevented the death of animals during the entire period of observation, only 22.2% (2 of 9) of the animals having died by day 14.

In the group of rats which received intraperitoneal nimodipine in a dose of 0.1 mg/kg, 20% of the rats died in the first 24 hours. By day 14 that figure was 40%.

The results obtained testify to the high protective activity of dimebon in relation to the basic stringent index of anti-insult action—preventing the death of rats after hemorrhagic insult. Dimebon is superior to nimodipine in relation to the ability to prevent the death of animals after insult.

A study of the neurological status of the surviving animals using the McGraw Stroke index showed that, in the group of animals with hemorrhagic insult, severe symptoms were observed in 50%, and mild symptoms in 87%, on the first day of observation (Table 4).

Dimebon reduced the neurological deficit in the animals, almost halving the number of animals with pareses. Nimodipine had a similar effect.

Dimebon thus had a positive effect in relation to the dynamics of development of neurological deficit in rats in the first days after hemorrhagic insult, and in relation to this effect was not inferior to nimodipine.

The studies performed have thus established that a pronounced neurological deficit and the death of animals are observed in rats with hemorrhagic insult. The pathological symptoms are observed to worsen by day 14 of observation. The dynamics of deterioration in the condition and death of rats with hemorrhagic insult testify to the latent insufficiency of compensatory reactions of the organism, increasing on specific critical days (3, 7, 14 days) of the post-operative period, and the development of concomitant complications (edema, swelling of tissues, disruption of intracerebral hemodynamics, elevated intracranial pressure, cerebral ischemia). When administered to animals 3 hours after insult and then for 14 days after the creation of hemorrhagic insult, dimebon has a marked anti-insult action, preventing the death of rats and weakening disturbance of the neurological status of the animals with post-traumatic hematoma. Dimebon is superior to nimodipine in the depth and extent of the effect.

TABLE 3
Effect of intraperitoneal administration of dimebon on
survival rate of animals after hemorrhagic insult (HI)

| | | 24 hours after | | 3 days after | | 7 days after | | 14 days after | |
|---|---|---|---|---|---|---|---|---|---|
| | | operation | | operation | | operation | | operation | |
| | | Number of animals dying during 14 days after hemorrhagic insult relative | | | | | | | |
| Groups of | Doses, | to total number of operated animals in absolute units and in % | | | | | | | |
| animals | mg/kg | a u | % | a u | % | a u | % | a u | % |
| Sham- | | 0/16 | 0 | 0/16 | 0 | 0/16 | 0 | 1/16 | 6.2 |
| operated | | | | | | | | | |
| Hemorrhagic | | 2/18 | 11.1* | 6/18 | 33.3* | 6/18 | 33.3% | 10/18 | 55.6* |
| insult | | | | | | | | | |
| Hemorrhagic | 0.1 | 0/9 | 0 | 1/9 | 11.1 | 1/9 | 11.1^{#} | 2/9 | 22.2^{#} |
| insult + | | | | | | | | | |
| Dimebon | | | | | | | | | |
| Hemorrhagic | 0.1 | 2/10 | 20 | 2/10 | 20 | 3/10 | 30 | 4/10 | 40 |
| insult + | | | | | | | | | |
| nimodipine | | | | | | | | | |

The reliability of the differences between the group of sham-operated animals and rats with HI-*- is (P≦0.05)(χ²); and between rats with control HI and those which received the preparations -#- is (P≦0.05)(χ²).

TABLE 4
Effect of dimebon on neurological deficit in rats after
hemorrhagic insult (HI), using the McGraw scale

| | Number of animals with neurological deficit 24 hours | | | |
|---|---|---|---|---|
| | after operation, in % | | | |
| Indices of | | Hemorrhagic | Hemorrhagic | |
| neurological | Hemorrhagic | insult + | insult + | Sham- |
| deficit | insult | Dimebon | nimodipine | operated |
| sluggish, | 87.5 | 88 | 50 | 33 |
| slow | | | | |
| movements | | | | |
| limb | 65 | 66 | 50 | 33 |
| weakness | | | | |
| circular | 12.5 | 33 | 25 | 0 |
| movements | | | | |
| paresis of | 50 | 33 | 25* | 0 |
| 1-4 limbs | | | | |
| | Number of animals with neurological deficit 14 days | | | |
| | after operation, in % | | | |
| Indices of | | Hemorrhagic | Hemorrhagic | |
| neurological | Hemorrhagic | insult + | insult + | Sham- |
| deficit | insult | Dimebon | nimodipine | operated |
| sluggish, | 12.5 | 7 | 16.6 | 6.6 |
| slow | | | | |
| movements | | | | |
| limb | 12.5 | 7 | 0 | 0 |
| weakness | | | | |
| circular | 0 | 0 | 0 | 0 |
| movements | | | | |
| paresis of | 0 | 0 | 0* | 0 |
| 1-4 limbs | | | | |

*Reliability of differences between rats with HI (control) and animals which had received the preparations (P ≦ 0.001) (χ²).

The results obtained testify that, along with its previously described properties, Dimebon can be used for the effective treatment of insult.

1. A method of treating insult in an individual in need thereof comprising administering to the individual a therapeutically effective amount of hydrogenated pyrido(4,3-b)indole of the formula (1) or a pharmaceutically acceptable salt thereof, 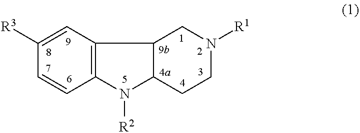
wherein:
R¹ is CH₃—, CH₃CH₂— or PhCH₂;
R² is H—, PhCH₂— or 6-CH₃-3-Py-(CH₂)₂—; and
R³ is selected from the group containing: H—, CH₃— or Br—.

2. The method of claim 1, wherein R¹ is CH₃—, R² is H—, and R³ is CH₃—.

3. The use as claimed in method of claim 2, wherein the compound is in the form of the (±)cis-isomer.

4. The use as claimed in method of claim 1, wherein the pharmaceutically acceptable salt is a pharmaceutically acceptable acid salt.

5. A method of treating insult in an individual in need thereof comprising administering to the individual a therapeutically effective amount of hydrogenated pyrido(4,3-b)-indole of the formula (2), or a pharmaceutically acceptable salt thereof, 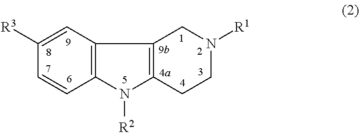
wherein:
R¹ is CH₃—, CH₃CH₂— or PhCH₃.
R² is H—, PhCH₂— or 6-CH₃-3-Py-(CH₂)₂—, and
R³ is selected from the group containing H—, CH₃— or Br—.

6. The method of claim 5, wherein R¹ is CH₃CH₂— or PhCH₂—, R² is H—, and R³ is H—.

7. The method of claim 5, wherein R¹ is CH₃—, R² is PhCH₂—, and R³ is CH₃—.

8. The method of claim 5, wherein R¹ is CH₃—, R² is 6-CH₃-3-Py-(CH₂)₂— and R³ is H—.

9. The method of claim 5, where R¹ is CH₃—, R² is 6-CH₃-3-Py-(CH₂)₂— and R³ is CH₃—.

10. The method of claim 5, wherein R¹ is CH₃—, R² is H—, and R³ is H— or CH₃—.

11. The method of claim 5, wherein R¹ is CH₃—, R² is H—, and R³ is Br—.

12. The method of claim 5, wherein the pharmaceutically acceptable salt is a pharmaceutically acceptable acid salt.

13. The method of claim 5, wherein the pyrido(4,3-b)-indole is 2,8-dimethyl-5-[2-(6-methyl-pyridyl-3)-ethyl]-2,3,4,5-tetrahydro-1H-pyrido[4,3-b]indole dihydrochloride.

14-15. (canceled)

16. A kit comprising: (1) a hydrogenated pyrido(4,3-b)-indole of the formula (2), or a pharmaceutically acceptable salt thereof 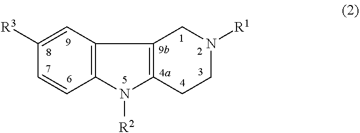
wherein:
R¹ is CH₃—, CH₃CH₂— or PhCH₃.
R² is H—, PhCH₂— or 6-CH₃-3-Py-(CH₂)₂—, and
R³ is H—, CH₃— or Br—; and
(2) instructions for use in the treatment of insult.

17. The kit of claim 16 wherein the hydrogenated pyrido(4,3-b)-indole is 2,8-dimethyl-5-[2-(6-methyl-pyridyl-3)-ethyl]-2,3,4,5-tetrahydro-1H-pyrido[4,3-b]indole dihydrochloride.
